(19) United States
(12) Patent Application Publication
(10) Pub. No.: US 2024/0408196 A1
(43) Pub. Date: Dec. 12, 2024

(54) ALKALINE EXTRACTION OF BETA GLUCAN COMPOUNDS FOR USE IN ANTI-VIRAL AND IMMUNE THERAPIES

(71) Applicants: Frank Jordan, Liberty, TX (US); Mark Campbell, Liberty, TX (US); Kenneth Hunter, Reno, NV (US); Sally Dupre, Reno, NV (US)

(72) Inventors: Frank Jordan, Liberty, TX (US); Mark Campbell, Liberty, TX (US); Kenneth Hunter, Reno, NV (US); Sally Dupre, Reno, NV (US)

(21) Appl. No.: 17/751,287

(22) Filed: May 23, 2022

Related U.S. Application Data
(60) Provisional application No. 63/281,869, filed on Nov. 22, 2021.
(60) Provisional application No. 63/192,399, filed on May 24, 2021.

(51) Int. Cl. A61K 39/39; A61K 39/00; A61P 37/04; C08B 37/00

(52) U.S. Cl. CPC A61K 39/39; A61P 37/04; C08B 37/0024; A61K 2039/542

(57) ABSTRACT

A method of extracting beta-glucan from autolyzed yeast material comprises a pre-wash step with water, an alkaline wash extraction step, a post-wash step with water, and if necessary, a drying step (e.g., spray-drying), with a dewatering and centrifugation between each step. The extraction improves efficiency by skipping the acidic and organic washes common to beta-glucan extraction. It has been discovered that beta glucan products yielded from this process have improved immunological properties, stimulating the expression of several immune-related genes in human macrophages without corresponding levels of stimulation for inflammatory pathways. The resulting beta glucan product can be administered as a supplement or adjuvant to existing anti-viral therapies.

### REFERENCE TO RELATED APPLICATIONS

This utility patent application claims priority to U.S. Provisional Application No. 63/192,399, filed on 24 May 2021 with the same inventors and title, and U.S. Provisional Application No. 63/281,869, filed on 22 Nov. 2021 with the same inventors and title. The contents of both provisional filings are included herein by reference.

### FIELD

Embodiments usable within the scope of the present disclosure relate, generally, to the autolysis and extraction of a specialized formulation of beta-(1,3)-linked and beta-(1,6)-linked glucopyranose (commonly and herein referred to as beta glucan). Specifically, strains of S. cerevisiae sourced from the food and beverage industry (e.g., baker's yeast, brewer's yeast), when extracted in accordance with the procedure described herein, produce unique biological response modifiers and results distinct from other sources of beta glucan.

### BACKGROUND

Glucans are polymers of glucose, commonly found in the cell walls of bacteria and yeast, as well as various other fungi and plant species. Two common glucans known as beta-(1,3)-linked glucopyranose and beta-(1,6)-linked glucopyranose (collectively “beta glucan” or “β-glucan”) have long been known to induce immuno-pharmacological activity in humans and animals.

In particular, beta glucan has been shown to potentiate and enhance key immune cell responses. For example, the human macrophage cell line THP-1, a common in vitro cell model, is stimulated by beta glucan, producing a variety of cytokines. These include, but are not limited to, TNF-α (a pro-inflammatory cytokine), MCP-1 (a chemotactic cytokine), IL-6 (which activates the acute phase response) and IL-10 (the prototypic immunosuppressive cytokine).

The immunologic activity of beta glucan has been a subject of study by researchers for several decades. While it is known to act on several immune receptors (e.g., CR3) and regulate the expression of several key glycoproteins, the mechanisms by which it stimulates the immune system are still not fully understood. The primary sources of beta glucan studied to date are baker's yeast, fungi, oats/barley, and algae. These sources can be utilized whole or as part of a cell wall extract.

A recurring challenge in stimulating the human immune system is to balance the increased production of these cytokines with the dangers of an overly aggressive inflammatory response (the so-called “cytokine storm”) which can be triggered by overly aggressive responses from both the body's natural immune system and synthetic treatments such as anti-viral medications.

Additionally, many viruses exhibit defense mechanisms which partially or completely block interferon synthesis or interferon action. Ordinarily, production of interferons enables the host cells to form a defense mechanism ensuring continued intercellular communication via paracrine signaling, which is generally obstructed by unchecked viral replication.

Prior art methods using beta glucan have emphasized solubilizing the product, often by means of a multi-step process primarily comprising a combination of alkali, acid, and organic extractions (e.g., alcohol), separated by washing steps, usually with water or buffered water. An example of such a soluble product can be found in U.S. Pat. No. 9,320,291 to Saarinen, et al. However, the particulate form of beta-glucan interacts with immunological receptors differently than the dissolved solute, since the particulate form is normally the one encountered in the wild (e.g., during a yeast infection).

Other methods have focused on insoluble particulate products, also with the same multi-step production, e.g., U.S. Pat. No. 6,476,003 to Jordan, et al., which discloses a manufacturing process to produce insoluble beta glucan products requiring similarly intensive multi-step processing. Additional physical (e.g., homogenization) and biological (e.g., enzymatic) methods have been used by others. However, these multi-step processes are time consuming and expensive.

A need therefore exists for a form of beta glucan which can maximize host immunity and antiviral protection and enhance the body's natural antiviral pathways without engendering a dangerous inflammatory response. A need also exists for a form of beta glucan which can be produced utilizing a simplified manufacturing method without the multi-step washing procedure used to produce currently available solubilized and insoluble beta glucan products.

Embodiments of the invention described herein meet this and other needs. Specifically, it has been discovered that a particulate beta glucan having strong modulating effects on components of the immune system can be obtained with a simplified procedure comprising an alkaline-only extraction of yeast cells, with water pre-wash and post-wash. The resulting product yields a beta glucan with not only equivalent but superior immune modulating capability, with omission of the acid and organic extractions and the associated washing steps eliminating approximately two-thirds of the time and expense.

### SUMMARY

The invention comprises a method of extraction for beta glucan comprising a three-step process of pre-washing, extraction, and post-washing, with each step followed by an intermediate centrifugation. In an embodiment, the process is completed with spray-drying step. The extraction step takes place in a highly alkaline environment with a pH level of at least 13.0. The resulting concentrated slurry comprises a high concentration of beta glucan derived from the cell walls of S. cerevisiae that appears to show increased expression of genes associated with the production of various interferons, as well as reduced activity at several genes associated with inflammation. This indicates a possible use for strains of S. cerevisiae used in the food & beverage industry combined with a novel simplified manufacturing process, yielding beta glucan compositions for use as orally consumed immune-modulating products, anti-viral nutritional supplements/therapies, chemotherapeutic agents, or vaccine adjuvants.

### BRIEF DESCRIPTION OF THE DRAWINGS

In the detailed description of the embodiments, presented below, reference is made to the accompanying drawings:

FIG. 1 depicts the two immunologically relevant forms of the beta glucan molecule.

FIG. 2 depicts the beta glucan molecules in a polymeric matrix structure.

FIG. 3 depicts a flowchart of an embodiment of a production method for preparing particulate beta glucan.

One or more embodiments are described below with reference to the listed Figures.

### DETAILED DESCRIPTION OF THE EMBODIMENTS

Before describing selected embodiments of the present disclosure in detail, it is to be understood that the present invention is not limited to the particular embodiments described herein. The disclosure and description herein is illustrative and explanatory of one or more presently preferred embodiments and variations thereof, and it will be appreciated by those skilled in the art that various changes in the design, organization, order of operation, means of operation, equipment structures and location, methodology, and use of mechanical equivalents may be made without departing from the spirit of the invention.

As well, it should be understood the drawings are intended to illustrate and plainly disclose presently preferred embodiments to one of skill in the art, but are not intended to be manufacturing level drawings or renditions of final products and may include simplified conceptual views as desired for easier and quicker understanding or explanation. As well, the relative size and arrangement of the components may differ from that shown and still operate within the spirit of the invention.

Moreover, it will be understood that various directions such as “upper,” “lower,” “bottom,” “top,” “left,” “right,” and so forth are made only with respect to explanation in conjunction with the drawings, and that the components may be oriented differently, for instance, during transportation and manufacturing as well as operation. Because many varying and different embodiments may be made within the scope of the concept(s) herein taught, and because many modifications may be made in the embodiments described herein, it is to be understood that the details herein are to be interpreted as illustrative and non-limiting.

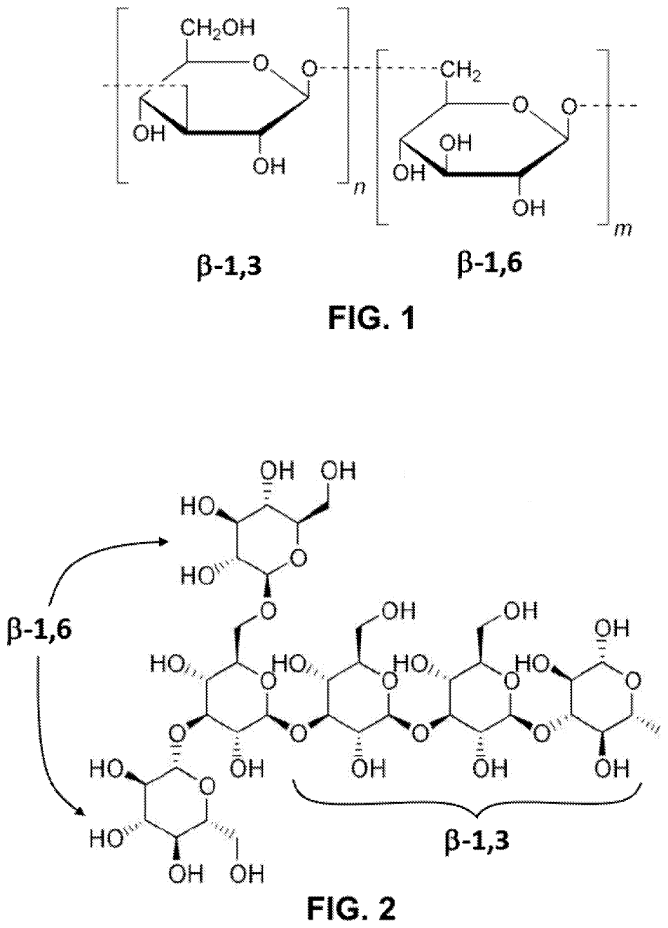

Turning first to FIGS. 1 and 2, the relevant beta glucan molecules are illustrated in isolation (FIG. 1) and as part of a polymeric cell wall matrix (FIG. 2). The typical yeast cell wall comprises a heavily branched core chain of β-1,3-glucan molecules with side chains terminating in β-1,6-glucan molecules. Prior art methods of extracting beta glucan often yield large, globular composites of beta glucan, while prior art efforts at forming a microparticulate preparation have often resulted in heavily breaking up the polymeric cell matrix, resulting in further formation of globular composites once subjected to the aqueous environments of the digestive system when taken orally.

Embodiments of autolysis and extraction discussed below minimize disruption to the cell wall matrix, retaining the three-dimensional structure of the beta glucan polymer to a greater extent than prior art beta glucan preparations, which is believed to provide the best opportunity for receptor interaction on hematopoietic cells, which have evolved to recognize the intact cell wall surface.

The standard extraction of beta-glucan from yeast cells involves a three-step process of an alkaline wash, an acidic wash, and an organic solvent wash. Examples of such multi-step processes are described in, e.g., U.S. Pat. Nos. 6,476,003 and 9,522,187. However, it has been discovered that a high-intensity alkaline wash alone may result in a product having superior immunologic activity.

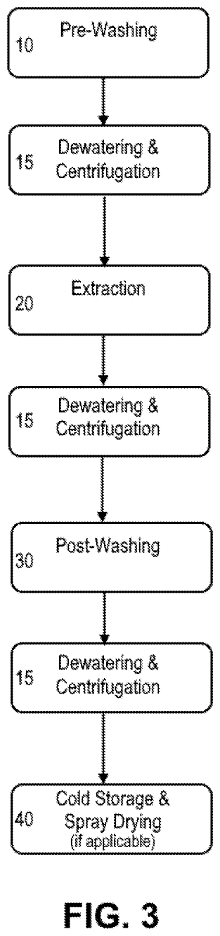

Turning now to FIG. 3, an embodiment of a manufacturing procedure is illustrated in the form of a stepwise flowchart. The pre-washing step 10 comprises 50-100 kg of autolyzed yeast material (added to a vat processor and hydrated with up to 1,900 L of water into a slurry. The goal of the pre-wash is to bring the slurry to a hydration level of up to 10.6%. The amount of material processed can vary depending on scale and operational equipment capacity.

To further facilitate pre-washing, the slurry may be heated as close as possible to the boiling point (100° C.). The pre-washing step 10 removes extraneous yeast extract remnants such as nucleotides, amino acids/peptides, fatty acids, and carbohydrates by up to 50%, thereby reducing the amount of extraction materials required in subsequent stages.

Subsequently, the extraction step 20 comprises an alkaline wash wherein the water is drained, leaving the prewashed solid material in vat processor, and water added to fill. The contents are heated to at least 80° C. and a food-grade alkaline extraction base (e.g., sodium hydroxide pellets) is added to bring the solution to a pH of no less than 13.0 for an extraction time of one hour. At the end of the extraction step 20, the solution is re-neutralized via a suitable food-grade acid (e.g., citric acid pellets).

Subsequent to the extraction step, the post-wash step 30 comprises draining and replacing the water to fill with the extracted solids material in the vat processor. As with the extraction step 20, the water may be added at ambient temperature or heated to a temperature of up to 80° C. in order to facilitate the process. The post-wash step 30 removes remaining sodium hydroxide as well as any intracellular molecules remaining from the extraction process.

After each stage of the process (pre-wash 10, extraction 20, and post-wash 30) the material is subject to an intermediate dewatering and concentration step 15 by means of centrifugation (either bench-top or continuous flow). Subsequent to the final dewatering and concentration step following the post-wash step 30, each batch can be composited into a lot and cold stored at 4° C. until, if appropriate for the needs an application of the final preparation, drying at an appropriate food-grade facility 40. The drying process, if necessary, may comprise spray drying, drum drying, fluid bed dryers, or other suitable technology which meets the needs of the end use product with minimal difference in recorded immunologic activity. In a preferred embodiment, spray-drying is used. Alternatively, the drying step may be omitted if the desired final preparation is, e.g., a liquid product.

The procedure described above and illustrated in FIG. 3 yields a high degree of pure beta glucan; the resulting dried material comprises roughly 80-90% by weight beta glucan. The primary remainders of the mixture comprise mannoproteins (10-20% by weight) and chitin (1-2% by weight).

Advantageously, the procedure yields a high quantity of beta glucan in its “native” cell wall configuration. As illustrated in FIG. 2, this configuration comprises a highly branched β-1,3-glucan backbone with side chains of β-1,6 glucan. The dry weight ratio of β-1,3-glucan to β-1,6-glucan in the resulting particulate is approximately 8:1.

In a further method embodiment, the beta glucan produced by the above-described process may be utilized in the various means listed, but not limited to, improving general immunity by means of nutritional or oral supplementing to normalize the immune cell responses, as a response to viral-initiated immune suppression, by enhancing the innate immune response to viral infections by re-establishing production of various interferons in infected epithelial cells.

When taken orally, the beta glucan compound may contribute to cessation of viral replication and re-establishment of paracrine signaling to non-infected epithelial cells, possibly resulting in infected patients incurring milder, less intense disease courses of shorter duration and contribute to lowered incidence rates of hospitalization.

### Examples

Table 1 depicts an example of the relative activity of four immunological mediators: beta glucan sourced from autolyzed S. cerevisiae yeast and produced in accordance with the above procedure, standard beta glucan sourced from baker's yeast and produced according to standard production methods, bacterial lipopolysaccharide (LPS), and polyinosinic: polycytidylic acid (poly I:C).

Human THP-1 monocytes growing in tissue culture were stimulated with an optimal of the two beta glucan preparations (5 particles per THP-1 cell), LPS (10 ng/ml), and poly I:C (10 ug/mL). After four hours, the RNA was extracted from both mediator-treated THP-1 cells and untreated control THP-1 cells and the change in gene expression measured utilizing a quantitative polymerase chain reaction (qPCR) assay. Results were recorded for the proinflammatory cytokines IL-6 and CXCL8, and the antiviral interferons IFN-α and IFN-β. The numbers in Table 1 represent the average-fold change in expression of the genes for each of the listed mediators, relative to untreated control cells.

TABLE 1
Gene Expression Effects of Beta Glucan on
Human THP-1 Monocytes (average fold increase)

| | Beta glucan | Beta | | |
|---|---|---|---|---|
| | (alkali-only | glucan | | |
| Mediator | wash) | (standard) | LPS | Poly I:C |
| IL-6 | 0.7 | 2.8 | 15 | 0.2 |
| CXCL8 | 0.8 | 1.14 | 30 | 0.8 |
| IFN-α | 5.8 | 4.0 | 3.7 | 2.8 |
| IFN-β | 7.3 | 1.8 | 7.1 | 1.6 |

As shown, both of the beta glucan preparations significantly upregulated the expression of genes for both interferons. As interferons are the primary early defense mechanisms against viral infection, this indicates the beta glucan preparations may stimulate antiviral immunity. Both beta glucans were more stimulatory than the Poly I:C, a common immunostimulant, and the beta glucan prepared with alkali-only wash showed higher stimulation of interferon than LPS.

LPS is known to cause fatal septic shock by overstimulating the production of inflammatory mediators such as IL-6 and CXCL8, as indicated by the very large multipliers for these cytokines. Compared to the LPS, both beta glucans resulted in significantly less stimulation of the cytokine receptors. As with the interferon production, this effect was magnified in the beta glucan prepared in accordance with the present methods.

The above test results indicate that beta glucan produced from autolyzed yeast via an alkali wash may stimulate general immunity as well as the antiviral mediators with less risk of cytokine overproduction than standard beta glucan sources. This beta glucan may be particularly suitable as a supplement for prophylaxis for immune support or therapy of viral infections, as the interferon response is non-specific and provides protection against almost all known viruses.

Additionally, interferon-producing drugs are often utilized in cancer treatment (e.g., chronic myeloid leukemia, metastatic breast cancer and prostate cancer) to stimulate a response from the body's immune system, indicating possible use for the beta-glucan as a supplement to chemotherapies.

There is also extensive research on the use of interferon-producing agents as vaccine adjuvants, for both cancer vaccines and vaccines for communicable diseases (e.g., flu vaccines) due to the utility of raising the body's natural immune response to the vaccine material, indicating that the beta-glucan may also possess potential as a vaccine adjuvant.

Table 2 depicts a further array of gene expression measurements utilizing qPCR to evaluate the expression of 85 genes coding for proteins involved in antiviral immunity. Duration and dosages were identical to those in Table 1. As with Table 1, the results are given in terms of average-fold increase in gene expression between the dosed and control THP-1 macrophages.

TABLE 2
Gene Expression Effects of Beta Glucan for Human
THP-1 Macrophages (average fold increase)

| | Beta glucan | Beta glucan | |
|---|---|---|---|
| Gene | (alkali-only wash) | (standard) | LPS |
| ARHGEF1 | 2.7 | 0.8 | 0.9 |
| ARHGEF10 | 2.4 | 0.8 | 0.7 |
| ARHGEF11 | 3.5 | 1.0 | 1.9 |
| ARHGEF12 | 2.2 | 0.9 | 2.2 |
| ARHGEF15 | 1.1 | 0.9 | 1.5 |
| ARHGEF2 | 2.4 | 0.7 | 1.7 |
| ARHGEF3 | 1.6 | 0.8 | 0.8 |
| ARHGEF4 | 7.8 | 8.4 | 15.9 |
| ARHGEF5 | 1.9 | 1.1 | 1.4 |
| ARHGEF6 | 2.9 | 0.8 | 0.7 |
| ARHGEF7 | 2.6 | 0.9 | 1.9 |
| ARHGEF9 | 1.1 | 0.9 | 15.8 |
| ATF4 | 1.5 | 1.2 | 1.5 |
| CREB1 | 1.8 | 1.0 | 1.7 |
| CREB3 | 2.3 | 1.4 | 3.1 |
| CREB3L4 | 1.3 | 0.7 | 0.2 |
| EIF4E | 1.4 | 0.8 | 1.0 |
| EIF4EBP1 | 1.3 | 0.9 | 0.1 |
| FRAP1 | 2.6 | 0.9 | 1.3 |
| H3F3B | 1.4 | 1.1 | 0.7 |
| HIST1H3A | 0.6 | 0.6 | 0.8 |
| HIST1H3B | 0.8 | 0.6 | 2.3 |
| HIST1H3C | 1.1 | 0.9 | 1.4 |
| HIST1H3D | 0.6 | 0.5 | 3.1 |
| HIST1H3F | 1.0 | 0.6 | 2.6 |
| HIST1H3G | 1.0 | 0.8 | 1.8 |
| HIST1H3H | 1.1 | 0.6 | 3.3 |
| HIST1H3I | 0.9 | 0.7 | 2.8 |
| HIST1H3J | 0.8 | 0.7 | 2.5 |
| HIST3H3 | 1.9 | 0.5 | 0.2 |
| IFNA14 | 4.8 | 1.7 | 0.3 |
| IFNA16 | 1.1 | 0.9 | 1.5 |
| IFNA17 | 1.3 | 0.1 | 0.2 |
| IFNA2 | 10.6 | 0.9 | 1.5 |
| IFNA4 | 0 | 0 | 0 |
| IFNA7 | 0.5 | 0 | 0.5 |
| IFNAR1 | 2.1 | 1.2 | 1.8 |
| IFNAR2 | 1.7 | 0.9 | 1.1 |
| IFNB1 | 1.9 | 1.4 | 4.2 |
| IFNG | 0 | 0 | 0 |
| IFNGRI | 2.1 | 1.1 | 2.7 |
| IFNGR2 | 1.6 | 2.0 | 5.7 |
| IFNW1 | 1.5 | 0.8 | 1.9 |
| IL6 | 0.3 | 2.7 | 18 |
| IRF9 | 1.1 | 1.0 | 4.7 |
| IRS1 | 1.1 | 0.6 | 3.4 |
| IRS2 | 3.6 | 0.8 | 0.3 |
| JAK1 | 2.1 | 0.9 | 1.8 |
| JAK2 | 2.3 | 0.9 | 1.4 |
| MAP2K3 | 2.3 | 2.1 | 5.7 |
| MAP2K6 | 1.0 | 0.3 | 0.2 |
| MAP3K1 | 3.1 | 0.7 | 0.8 |
| MAP3K2 | 2.5 | 1.1 | 2.3 |
| MAP3K3 | 2.4 | 0.9 | 1.6 |
| MAP3K4 | 3.1 | 0.8 | 1.4 |
| MAP3K5 | 2.9 | 0.9 | 6.3 |
| MAP3K6 | 2.4 | 0.6 | 0.6 |
| MAPK11 | 2.1 | 0.9 | 5.0 |
| MAPK12 | 2.5 | 0.8 | 0.7 |
| MAPK13 | 2.8 | 0.8 | 3.7 |
| MAPK14 | 2.6 | 0.7 | 1.1 |
| MCF2L | 1.1 | 0 | 1.5 |
| NET1 | 2.9 | 7.1 | 16.5 |
| PIK3C2A | 2.8 | 0.7 | 1.3 |
| PIK3C2B | 3.0 | 0.6 | 0.6 |
| PIK3C3 | 1.0 | 0.8 | 1.3 |
| PIK3CA | 2.5 | 0.8 | 2.2 |
| PIK3CB | 1.4 | 0.8 | 0.2 |
| PIK3CD | 2.8 | 1.0 | 1.7 |
| PIK3R1 | 2.4 | 0.7 | 1.6 |
| PIK3R2 | 2.5 | 0.9 | 0.4 |
| PIK3R3 | 2.1 | 0.8 | 1.7 |
| PIK3R4 | 1.5 | 0.6 | 0.8 |
| PIK3R5 | 3.2 | 1.1 | 17.4 |
| PRKCD | 1.5 | 1.3 | 1.9 |
| RAC1 | 1.2 | 1.0 | 1.7 |
| RPS6 | 1.0 | 1.0 | 0.8 |
| RPS6KA4 | 3.0 | 0.7 | 0.3 |
| RPS6KA5 | 1.8 | 0.6 | 1.2 |
| RPS6KB1 | 2.1 | 0.9 | 1.5 |
| STAT1 | 2.3 | 1.2 | 4.6 |
| STAT2 | 2.4 | 0.6 | 4.3 |
| TYK2 | 3.1 | 0.8 | 3.2 |
| VAV1 | 1.7 | 0.7 | 1.8 |
| VAV2 | 3.0 | 0.9 | 1.5 |
| VAV3 | 2.0 | 0.9 | 2.7 |

As shown in Table 2, the beta glucan produced via alkali wash triggers a significantly broader spectrum of immunological gene expression compared with either the standard preparation of beta glucan or the LPS. Over 40 genes in the list of Table 2 show a doubled expression compared with only 5 genes for the standard beta glucan. In particular, the beta glucan prepared in accordance with the above-described methods resulted in significant upregulation of several interferon genes (e.g., IFNA2, IFNA14) compared to standard beta glucan, a further indication of the potential of this beta glucan for antiviral therapies. In addition, the alkali-only preparation of beta glucan resulted in much lower stimulation of known inflammation pathways (e.g., IL6) than the LPS preparation (such stimulation often ruling out LPS as an anti-viral).

Although several preferred embodiments of the invention have been illustrated in the accompanying drawings and described in the foregoing specification, it will be understood by those of skill in the art that additional embodiments, modifications and alterations may be constructed from the invention principles disclosed herein, while still falling within the scope of the disclosed invention.

1. A method of preparing particulate beta-glucan from yeast comprising:
hydrating autolyzed yeast material with water in a vat processor;
draining and replacing the water;
extracting solid material from the slurry by adding an alkaline extraction base sufficient to bring the pH to between 10 and 14 for a period of between 5 minutes to 2 hours; and
neutralizing the slurry and extracted solid material.

2. The method of claim 1, further comprising the step of washing the extracted solid material in the vat processor with another fill of water.

3. The method of claim 2, wherein the step of extracting solid material, the step of washing the extracted solids material, or both, are conducted at a temperature of up to 80° C.

4. The method of claim 1, wherein the step of hydrating the autolyzed yeast material is conducted at a temperature of up to 100° C., at a ratio sufficient to produce a slurry having a hydration level up to 10.6%.

5. The method of claim 1, further comprising the step of dewatering and concentrating the extracted solid material by centrifugation, hydroclones, sedimentation, and/or filtration.

6. The method of claim 5, further comprising the step of drying the extracted solid material and cold-storing at 4° C.

7. The method of claim 6, wherein the step of drying the extracted solid material comprises spray-drying, drum-drying, barrel-drying, or fluid-bed drying.

8. The method of claim 1, wherein the extracted solid material comprises at least 80% beta-glucan by weight.

9. The method of claim 8, wherein the beta-glucan results in an increase in the expression of immune-enhancing cytokines and/or interferon-producing genes by human macrophages.

10. A method of using the beta-glucan produced by the method of claim 1 wherein the beta-glucan comprises an adjuvant for general immune enhancement, anti-viral or anti-cancer treatments.

11. A method of administering vaccinations comprising the use of beta-glucan produced by the method of claim 1 as an adjuvant.

12. A method of increasing the expression of interferon-producing genes by human macrophages comprising:
bringing a slurry of autolyzed yeast material to an alkaline pH to extract an immunologically active beta-glucan;
directly neutralizing the immunologically active beta-glucan from the alkaline slurry; and
orally administering the particulate alone or in conjunction with an existing anti-viral treatment.

13. The method of claim 12, further comprising the step of creating the slurry of autolyzed yeast material by hydrating autolyzed yeast material with water in a vat processor at a ratio sufficient to produce a slurry having a hydration level up to 10.6%.

14. The method of claim 12, wherein the step of bringing the slurry to an alkaline pH is conducted at a temperature of up to 80° C.

15. The method of claim 12, wherein the step of extracting the immunologically active beta-glucan consists of an alkaline wash.

16. The method of claim 12, further comprising the step of drying the extract to produce a particulate material subsequent to neutralizing the beta-glucan from the slurry.

17. The method of claim 16, wherein the step of drying the extracted solid material comprises spray-drying, drum-drying, barrel-drying, or air-drying.
